(19) United States
(12) Patent Application Publication
(10) Pub. No.: US 2012/0035570 A1
(43) Pub. Date: Feb. 9, 2012

(54) FEMININE HYGIENE APPARATUS AND METHOD

(76) Inventor: Gwendolyn Washington, Glen Burnie, MD (US)

(21) Appl. No.: 13/098,560

(22) Filed: May 2, 2011

Related U.S. Application Data
(60) Provisional application No. 61/370,592, filed on Aug. 4, 2010.

(51) Int. Cl. A61F 13/84

(52) U.S. Cl. 604/385.02

(57) ABSTRACT

An apparatus comprising a first feminine sanitary napkin, and a bag having an inner chamber and resealable means which can be unsealed to allow the inner chamber to be accessed and can be sealed to prevent access to the inner chamber. The bag may be attached to the first feminine sanitary napkin; and the bag may be impermeable or substantially impermeable to liquids. The apparatus may also include a second feminine sanitary napkin. The bag may be attached to the first feminine sanitary napkin and to the second feminine sanitary napkin so that the bag is between the first feminine sanitary napkin and the second sanitary napkin.

### CROSS REFERENCE TO RELATED APPLICATION(S)

The present application claims the priority of U.S. provisional patent application Ser. No. 61/370,592, titled “2 in 1 Mini-Maxi Pad”, filed on Aug. 4, 2010.

### FIELD OF THE INVENTION

This invention relates to improved methods and apparatus concerning feminine hygiene products.

### BACKGROUND OF THE INVENTION

Many women are required to carry a plurality of feminine hygiene products when leaving the home. These feminine hygiene products may include a plurality of maxi pads, feminine sanitary towels, feminine sanitary napkins or other absorbent pieces of material typically worn by a woman during her period to absorb her menstrual flow.

Carrying around a plurality of feminine hygiene products, such as a plurality of maxi pads, can be inconvenient, as women may have limited storage space in their purses, bags, or pockets for containing these products. Also, many occasions are not conducive to carrying purses or bags. While golfing, dining at restaurants, or doing various activities, purses may be a hindrance, or they may not be allowed at all. This leaves women with no way to carry hygienic products, and thus prevents women from using them as they are needed. An effective solution is needed.

### SUMMARY OF THE INVENTION

At least one embodiment of the present invention provide a two-in-one mini or maxi pad apparatus which provides women with a convenient and effective feminine hygiene product that eliminates the need for carrying multiple products at once. In at least one embodiment a maxi pad apparatus containing two maxi pads is provided. One of the maxi pads may be removed and discarded to reveal a fresh, new maxi pad underneath. The two maxi pads can be separated by protective, adhesive strips, and may contain a sealed bag, container, enclosure, or packet of moistened wipes to allow users to cleanse themselves before utilizing the next fresh maxi pad. Women will enjoy the discreet convenience and freshness offered by an apparatus of one embodiment of the present invention.

In at least one embodiment, an apparatus is provided comprising a first feminine sanitary napkin, and a bag having an inner chamber and resealable means which can be unsealed to allow the inner chamber to be accessed and can be sealed to prevent access to the inner chamber. The bag may be attached to the first feminine sanitary napkin; and the bag may be impermeable or substantially impermeable to liquids.

The apparatus may also include a second feminine sanitary napkin. The bag may be attached to the first feminine sanitary napkin and to the second feminine sanitary napkin so that the bag is between the first feminine sanitary napkin and the second sanitary napkin.

The apparatus may include a moistened material stored within the inner chamber of the bag. The moistened material can be removed from the inner chamber of the bag when the resealable means is unsealed.

A method is also provide which may include attaching an apparatus to a female person's underwear. The apparatus may be as previously described. The method may also include unsealing the resealable means and removing the moistened material from the inner chamber of the bag when the resealable means is unsealed.

To the accomplishment of the foregoing and related ends, certain illustrative aspects are described herein in connection with the following description and the annexed drawings. These aspects are indicative of the various ways in which the principles disclosed herein can be practiced and all aspects and equivalents thereof are intended to be within the scope of the claimed subject matter. Other advantages and novel features will become apparent from the following detailed description when considered in conjunction with the drawings.

### BRIEF DESCRIPTION OF THE DRAWINGS

FIG. 1 shows a top, right, front, perspective view of an apparatus in accordance with the present invention with the apparatus in an assembled state;

FIG. 2 shows a top view of the apparatus of FIG. 1, with the apparatus of FIG. 1 in the assembled state;

FIG. 3 shows a right side view of the apparatus of FIG. 1, with the apparatus of FIG. 1 in the assembled state;

FIG. 4 shows a right side view of the apparatus of FIG. 1, with the apparatus of FIG. 1 shown taken apart in a disassembled state; and

FIG. 5 shows a top, right, front perspective view of the apparatus of FIG. 1, with the apparatus shown taken apart in a disassembled state.

### DETAILED DESCRIPTION OF THE DRAWINGS

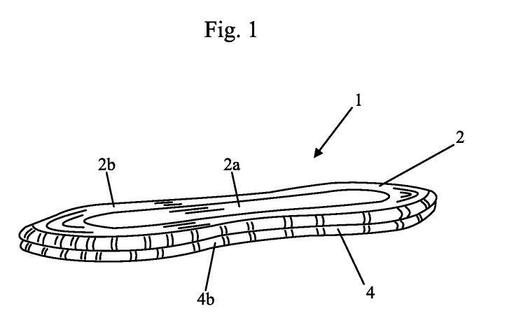

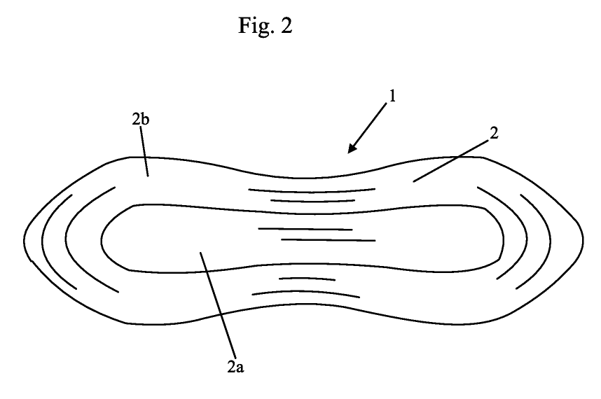

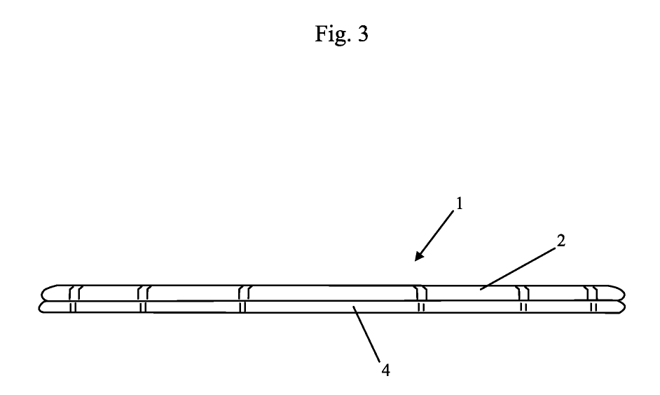

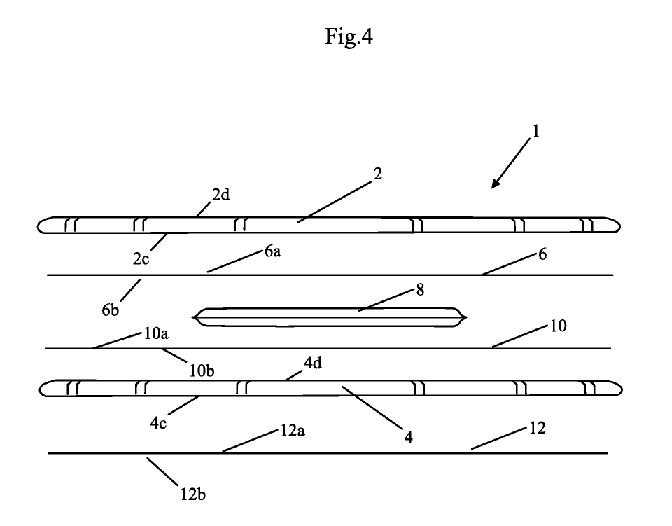

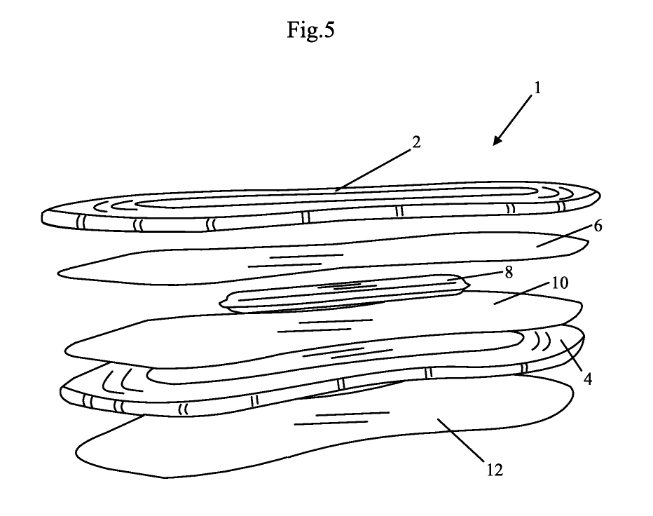

FIG. 1 shows a top, right, front, perspective view of an apparatus 1 in accordance with the present invention with the apparatus 1 in an assembled state. FIG. 2 shows a top view of the apparatus 1, with the apparatus 1 in the assembled state. FIG. 3 shows a right side view of the apparatus 1, with the apparatus 1 in the assembled state. FIG. 4 shows a right side view of the apparatus 1, with the apparatus 1, shown taken apart in a disassembled state. FIG. 5 shows a top, right, front perspective view of the apparatus 1, with the apparatus 1 shown taken apart in a disassembled state.

The apparatus 1 includes a first pad 2, a first protective adhesive strip 6, a bag, enclosure, container, or packet 8, a second protective adhesive strip 10, a second pad 4, and a protective backing 12 as shown in FIGS. 4 and 5.

Each of the first pad 2 and the second pad 4 may be a feminine maxi pad, feminine sanitary towel, feminine sanitary napkin or other absorbent piece of material typically worn by a woman during her period to absorb her menstrual flow. Each of the first pad 2 and the second pad 4 may be made out of any known feminine maxi pad, such as for example a super-absorbent material covered by quilted cotton. Each of the first pad 2 and the second pad 4 may be made of traditional or biodegrable, absorbent material suitable for construction of feminine maxi pads.

The bag 8 may be a non-porous bag such as non-porous plastic bag, which may contain moistened wipes or moistened clothes. The bag 8 may be impermeable to liquids, and/or solids, and/or any other materials. The moistened wipes or clothes may be sealed within the non-porous bag 8.

Each of the strips 6, 10, and 12 may be made of paper of some other material and may have adhesive on both a top surface and a bottom surface. For example, strip 6 has adhesive on top surface 6a for attaching to bottom surface 2c of pad 2 and adhesive on bottom surface 6b for attaching to bag 8 and top surface 10a of strip 10. Strip 10 has adhesive on top surface 10a for attaching to bag 8 and surface 6b of strip 6, and adhesive on bottom surface 10b for attaching to top surface 4d of pad 4. Strip 12 has adhesive on top surface 12a for attaching to bottom surface 4c of pad 4. The strip 12 may not have adhesive on the bottom surface 12b, if the strip 12 is removed and the bottom surface 4c of the pad 4 is attached directly to underwear of a woman.

The protective backing 12 can be removed from the second pad 4 to reveal an adhesive on the surface 4c of the pad 4. The surface 4c can then be attached to a woman's underwear to attach the apparatus 1, except for the removed backing 12, to the woman's underwear.

After the first pad 2 has been used (i.e. has absorbed sufficient menstrual flow, such as through the surface 2d), then the first pad 2 is removed. The adhesive strip 6 is also removed at that time to reveal the bag 8. The bag 8, may be a sealable plastic bag, or other sealable container, which can be removed from the second pad 4, opened or unsealed to take out moistened wipes or clothes inside of the bag 8. The moistened clothes (not shown) can then be used by the woman.

Finally the adhesive strip 10 can also be removed so that the pad 4 can be used to absorb menstrual flow of the woman, such as through the top surface 4d.

The apparatus 1 provides women with a discreet and effective feminine hygiene product.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. An apparatus comprising:
a first feminine sanitary napkin; and
a bag having an inner chamber, wherein the inner chamber is sealed within the bag so that there is no access to the inner chamber unless the bag is unsealed allowing access to the inner chamber; and
wherein the bag is attached to the first feminine sanitary napkin; and
wherein the bag is substantially impermeable to liquids.

2. The apparatus of claim 1 wherein
the bag includes a resealable means which can be unsealed to allow the inner chamber to be accessed and can be sealed to prevent access to the inner chamber.

3. The apparatus of claim 1 further comprising
a second feminine sanitary napkin; and
wherein the bag is attached to the first feminine sanitary napkin and to the second feminine sanitary napkin so that the bag is between the first feminine sanitary napkin and the second sanitary napkin.

4. The apparatus of claim 1 further comprising
a moistened material stored within the inner chamber of the bag; and
wherein the moistened material can be removed from the inner chamber of the bag when the resealable means is unsealed.

5. The apparatus of claim 3 further comprising
a moistened material stored within the inner chamber of the bag; and
wherein the moistened material can be removed from the inner chamber of the bag when the resealable means is unsealed.

6. A method comprising the steps of
attaching an apparatus to a female person's underwear;
wherein the apparatus includes
a first feminine sanitary napkin; and
a bag having an inner chamber, wherein the inner chamber is sealed with the bag so that there is no access to the inner chamber unless the bag is unsealed allowing access to the inner chamber; and
wherein the bag is attached to the first feminine sanitary napkin; and
wherein the bag is substantially impermeable to liquids.

7. The method of claim 6 wherein
the bag includes a resealable means which can be unsealed to allow the inner chamber to be accessed and can be sealed to prevent access to the inner chamber.

8. The method of claim 6 wherein
the apparatus is further comprised of:
a second feminine sanitary napkin; and
wherein the bag is attached to the first feminine sanitary napkin and to the second feminine sanitary napkin so that the bag is between the first feminine sanitary napkin and the second sanitary napkin.

9. The method of claim 6 wherein
the apparatus is further comprised of a moistened material stored within the inner chamber of the bag; and
further comprising unsealing the bag; and
removing the moistened material from the inner chamber of the bag when the bag is unsealed.

10. The method of claim 7 wherein
the apparatus is further comprised of a moistened material stored within the inner chamber of the bag; and
further comprising unsealing the resealable means and
removing the moistened material from the inner chamber of the bag when the resealable means is unsealed.
